(19) United States
(12) Patent Application Publication
(10) Pub. No.: US 2007/0014850 A1
(43) Pub. Date: Jan. 18, 2007

(54) Process for the preparation of dispersible tablets of cephalexin

(76) Inventors: Ramalingam Manikandan, CHINGLEPUT, TAMIL NADU (IN); Ashish Gogia, Delhl (IN); Sunilendu Bhushan Roy, Mumbai, Maharashtra (IN); Rajlv Malik, Wlen (AT)

Correspondence Address: Jayadeep R. Deshmukh;Ranbaxy Inc., 600 College Road East, Suite 2100, Princeton, NJ, 08901, US

(21) Appl. No.: 10/523,116

(22) PCT Filed: Aug. 1, 2003
(86) PCT No.: PCT/IB03/03092

§ 371 Date: Mar. 10, 2006

(30) Foreign Application Priority Data
Aug. 2, 2002 (IN) 815/DEL/2002

(51) Int. Cl. A61K 9/20; B27N 3/00

(52) U.S. Cl. 424/464; 264/109

(57) ABSTRACT

The present invention relates to dispersible tablets of cephalexin and a process for their preparation.

### FIELD OF THE INVENTION

The present invention relates to dispersible tablets of cephalexin and a process for their preparation.

### BACKGROUND OF THE INVENTION

Cephalexin [7-D-α-Amino-α-phenylacetamido)-3-methyl-3-cephem-4-carboxylic acid] belongs to the class of cephalosporin β-lactam antibiotics. It is a semisynthetic cephalosporin antibiotic intended for oral administration. Cephalexin has been shown to be active against a variety of gram positive and gram negative bacteria. Presently, cephalexin is available as capsules, tablet and dry syrup.

The major drawback for a tablet dosage form is that they are large in size and often are difficult for pediatric and geriatric patients to swallow. Further, there is also the problem of dissolution and disintegration of these tabletted formulations, which is a prerequisite of any formulation to achieve an effective plasma concentration of the particular active pharmaceutical ingredient. The absorption of a medicament from a dosage form should be both fast and predictable. Suspension formulations have been found to be better candidates, as compared to tablets, due to the human body's rapid absorption of drugs in such a dosage form.

Dry syrups present additional problems, in that they need to be reconstituted with water before ingestion. These formulations can be bulky and require accurate measurement tools to deliver the correct dose, which is not always conducive to patient compliance. Normally, suspensions are refrigerated to prevent the loss of potency and therefore are inconvenient while traveling. Further, skills for precise measurement of dose of the correct dose are required.

Water dispersible tablets solve some of the above-mentioned problems. Prior art describes some compositions and methods of preparation of dispersible tablet for amoxicillin and cefaclor antibiotics.

For example, U.S. Pat. No. 4,950,484 describes a dispersible tablet of amoxicillin comprising a mixture of microcrystalline cellulose and low substituted

For example, U.S. Pat. No. 4,950,484 describes a dispersible tablet of amoxicillin comprising a mixture of microcrystalline cellulose and low substituted hydroxypropyl cellulose as disintegrants. Similarly, U.S. Pat. No. 5,681,141 describes a process for preparation of dispersible tablets of cefaclor by direct compression comprising a disintegrant, and sodium stearyl fumarate as a lubricant. U.S. Pat. No. 5,861,172 provides a process for the manufacture of a tablet in which granules comprising a compacted mixture of amoxicillin, together with an intra-granular disintegrant, are mixed with an extra-granular disintegrant to form a tablet. U.S. Pat. No. 5,837,292 provides a granulate comprising a beta-lactam antibiotic in a mixture with a water dispersible cellulose such as microcrystalline cellulose and/or sodium carboxymethylcellulose.

U.S. Pat. No. 5,955,107 describes a pharmaceutical suspension tablet comprising antibiotics, croscarmellose sodium, microcrystalline cellulose and a co-processed additive consisting essentially of microcrystalline cellulose and calcium, sodium alginate complex.

Finally, U.S. Pat. No. 4,886,669 discloses a water dispersible tablet consisting of coated microparticles of antibiotics, disintegrants and a swellable material.

None of the above prior art provides a simple and easy method of manufacturing a water-dispersible dosage form of cephalexin in particular. Further, the primary requisite of a dispersible tablet is that it should rapidly disintegrate in water, forming a uniform suspension that has a smooth mouth feel without any gritty particles.

### SUMMARY OF THE INVENTION

In one general aspect there is provided a process for preparing a water dispersible tablet of cephalexin which disintegrates within 3 minutes in water at 20° C.±5° C. to form a uniform suspension. The process includes granulating cephalexin, disintegrant(s), and colloidal silicon dioxide with a binder solution; drying the resulting granules; mixing with disintegrant(s), fillers, lubricating agents and other optional excipients; and compressing to form tablets. Further, in this general aspect, the dispersible tablet includes cephelaxin monohydrate and includes particles having a particle size of less than 250 μm of cephalexin.

In another general aspect, there is provided a water dispersible dosage form of cephalexin comprising an intragranular and an extragranular portion. The intragranular portion includes cephalexin and its pharmaceutically acceptable salts, disintegrant(s), and suspending agent(s). The extragranular portion comprises one or more pharmaceutically acceptable excipients.

The dispersible tablet may include one or more disintegrants including sodium starch glycolate, carboxy methylcellulose, croscarmellose sodium and crospovidone and combinations thereof. In the preferred embodiment, crospovidone is used in an amount ranging from about 0.5% to about 10% by weight of the total weight.

The dispersible tablet may include one or more binders including hydroxypropyl cellulose, hydroxypropyl methylcellulose, polyvinylpyrrolidone and combinations thereof. In the preferred embodiment, polyvinyl pyrrolidone is used in an amount ranging from about 0.25% to about 4% by weight of the total tablet weight.

This dispersible tablet may also include one or more fillers including lactose, microcrystalline cellulose, mannitol, and combinations thereof. In the preferred embodiment the filler is either mannitol or microcrystalline cellulose.

The dispersible tablet may also include one or more lubricants including magnesium stearate, stearic acid, sodium stearyl fumarate and combinations thereof. In the preferred embodiment the lubricant is magnesium stearate used in an amount that ranges from about 0.25% to about 5% by weight of the total tablet weight.

Embodiments of this dosage form may further include one or more of the following features. For example, suspending agents, sweeteners, coloring agents, antiadherants, and flavoring agents.

The dispersible tablet also may include one or more suspending agents including microcrystalline cellulose, sodium carboxy methylcellulose, colloidal silicon dioxide, mannitol, povidone, sodium starch glycolate or a combination thereof. In the preferred embodiment the suspending agent is colloidal silicon dioxide used in an amount ranging from about 0.25% to about 6.0% by weight of total tablet weight.

The dispersible tablet also may include one or more sweeteners including sugars, saccharin or its salts, aspartame or combinations thereof. In the preferred embodiment the sweetener is aspartame used in an amount ranging from about 0.01% to about 2.0% by weight of total weight of tablet.

In the preferred embodiment, the dispersible tablet may also include optional ingredients, including the coloring agent D & C Yellow Aluminum Lake, the antiadherant colloidal silicon dioxide, and the flavor agent peppermint.

In another general aspect, this invention relates to a method of treating an infection in a human caused by microorganisms susceptible to cephalexin comprising providing cephalexin in the form of a water dispersible tablet as described.

The dispersible tablets produced by this process are stable for at least three months at accelerated stability conditions of 40° C./75% RH.

### DETAILED DESCRIPTION

The invention arises from the discovery that water dispersible tablets of Cephalexin, which disintegrate within 3 minutes in water at 20° C.±5° C. to form a uniform suspension of cephalexin, can be easily prepared by the wet granulation method utilizing an optimum amount of disintegrant, colloidal silicon dioxide and binder(s).

Therefore, in one aspect, herein is provided a process for the preparation of water dispersible tablets of Cephalexin, which disintegrate within 3 minutes, in water at 20° C.±5° C., to form a uniform suspension.

In another aspect, a process for the preparation of a water dispersible tablet of cephalexin is provided. The mixture of cephalexin, disintegrant and colloidal silicon dioxide are then granulated with a binder solution. The resulting granules are dried and mixed with disintegrants, fillers, lubricating agents and, optionally, other excipients. This mixture is then compressed into tablets.

In addition, granules of the present invention may also comprise suspending agents and/or coloring agents. Optionally, other excipients may be selected from antiadherants, sweeteners, coloring agents and flavoring agents.

The dispersible tablets of the present invention readily disperse in water in less than three minutes, giving a uniform suspension, which is free of grit and lumps. The suspension formed by dispersing two tablets in 100 ml of water has a particle size distribution of d₉₀ less than 600 μm. The cephalexin particles remain suspended for a sufficient period of time for easy dosing.

For the purpose of present invention Cephalexin is present as cephalexin monohydrate.

The particle size of cephalexin used in accordance with the present invention was reduced to d₉₀ less than 250 μm. The amount of cephalexin may vary from about 20% to about 50% by weight of the total tablet weight.

The disintegrants of the present invention may comprise one or more of sodium starch glycolate, carboxy methylcellulose, croscarmellose sodium and crospovidone or combinations thereof. The disintegrant may be used in an amount from about 0.5% to about 10% w/w. The intragranular and extragranular disintegrants may be the same or different. The preferred disintegrant is crospovidone.

Binders of the present invention may comprise one or more of hydroxypropyl cellulose, hydroxypropyl methylcellulose and polyvinylpyrrolidone (povidone) or combinations thereof. The binder of the present invention may be present in an amount from about 0.25% to about 4% by weight of the total weight of tablet. The preferred binder is polyvinylpyrrolidone. The ratio of the amount of disintegrant to the amount of binder is chosen to obtain fast-dispersing tablets having less friability. For the purpose of the present invention the ratio of the amount of disintegrant to the amount of binder from 1:1 to 1:20 depending upon the disintegrant and binder used, for example, 1:5 to 1:15 is the preferred level.

The fillers of the present invention may comprise one or more of lactose, microcrystalline cellulose, mannitol or combinations thereof The preferred diluent is microcrystalline cellulose, which also acts as both a binder and disintegrant by virtue of its swelling properties. Various types of commercially available microcrystalline cellulose can be used, and a particular type can be either AVICEL PH 101, for example having average particle size of about 50 microns, or AVICEL PH 302, for example having average particle size of about 90 microns.

The suspending agent of the present invention may be selected from the group consisting of microcrystalline cellulose, sodium carboxy methyl cellulose, colloidal silicon dioxide, mannitol, povidone, sodium starch glycolate, veegum or combinations thereof.

The lubricants of the present invention may comprise one or more of magnesium stearate, stearic acid, sodium stearyl fumarate or combinations thereof. A particular lubricant can be magnesium stearate. The lubricant may be used in an amount of about 0.25% to about 5% by weight of total tablet weight.

For the purpose of this invention, colloidal silicon dioxide also includes colloidal silica or its derivatives such as Syloid. Colloidal silicon dioxide serves two purposes, first as antiadherant and then as a suspension aid. It can be used intragranularly as well extragranularly. A particular amount of colloidal silicon dioxide can be from about 0.25% to about 6.0% by weight of the total tablet weight.

Sweeteners for the present invention may comprise one or more of sugars, saccharin or its salts, aspartame or combinations thereof. The amount used may depend upon the sweetener used. A particular sweetener can be aspartame, at about 0.01% to about 2.0% by weight of the total tablet weight.

For this formulation any flavoring agent approved by FDA for oral use may be used. Particular flavors can be “Flavor Peppermint” and “Flavor fruit gum”. A particular amount of flavoring agent can be from about 0.1% to 4.0% by weight of the total formulation weight.

Colorants impart aesthetics and the preferred choice is D&C Yellow Aluminum Lake at less than 1% w/w of the formulation. These may be used intragranularly or extragranularly.

The dispersible tablet of the present invention can be prepared by a wet granulation method. Such methods result in more porous granules which aid in rapid disintegration. Low particle size of the excipients in a suspension made from a dispersible tablet, is directly correlated to a smooth mouth feel. As per British Pharmacopoeia, the particles of a suspension should pass through a 600 μm sieve without leaving any residue. A suspension complying with this requirement can, however, still have a gritty mouth feel. Therefore, it can be desirable to have a finer suspension containing a more uniform size particles. The dispersible tablets made in accordance with the present invention form a uniform dispersion upon swirling which has a smooth mouth feel and is free of gritty particles. The particle size distribution in the suspension was d₉₀ less than 600μm.

The cephalexin, colloidal silicon dioxide, coloring agent and disintegrant are all sifted. Next, the sifted cephalexin, color (half quantity), and disintegrant (half quantity) are mixed in a Rapid Mixer Granulator. Binder is sifted and dissolved in a measured quantity of purified water using a mechanical stirrer. The premix is then wet granulated with the binder solution in a rapid mixer granulator. The granules are dried in a fluidized bed dryer at 60°±5° C. The dried granules are sifted through mesh 22 BSS (699 μm) and collected. Fillers, antiadherant, colorant and disintegrant are sifted and mixed in a non-shear blender. The dried granules are then mixed with the premix of filler, antiadherant, colorant and disintegrant in a non-shear blender for 20 minutes. Sweetener and flavor are sifted through a mesh 60 BSS (251 μm) sieve and added to the above blend and mixed for 5 minutes. Finally, lubricant is added and blended for 10 minutes. Next, the blend is compressed with appropriate tooling to make tablets.

The dispersible tablets of the present invention maintain the same advantages as conventional tablets and capsules in terms of their accuracy of dosing and ease of handling. They also possess the advantages of suspensions in terms of better bioavailability and increased compliance with children, elderly and patients who have difficulty in swallowing. These tablets have low friability and therefore are easily transportable. As opposed, to a suspension, no refrigeration is required. The dispersible tablets of the present invention are meant to form a suspension and can also be administered as conventional tablet. Additionally, the granules that are compressed to form these tablets can be used to form rapidly disintegrating chewable tablets or lozenges.

The following example illustrates specific embodiments of the invention and do not limit it.

### EXAMPLE 1

| COMPONENT | WEIGHT (mg) | % by weight |
|---|---|---|
| Intragranular | | |
| Cephalexin | 264.02 | 33.00 |
| Crospovidone | 12.00 | 1.50 |
| Colloidal silicon dioxide | 12.0 | 1.50 |
| Povidone | 4.00 | 0.50 |
| D&C Yellow 10 aluminum lake | 0.26 | 0.0325 |
| Purified water | qs | qs |
| Extragranular | | |
| Mannitol | 180.0 | 22.50 |
| Microcrystalline cellulose | 254.72 | 31.84 |
| Crospovidone | 36.00 | 4.50 |
| Aspartame | 10.00 | 1.25 |
| Flavour Peppermint 517 | 2.00 | 0.25 |
| Flavor Fruit Gum 912 | 10.00 | 1.25 |
| D&C Yellow 10 aluminum lake | 1.00 | 0.125 |
| Colloidal silicon dioxide | 4.00 | 0.50 |
| Magnesium stearate | 10.00 | 1.25 |

Process:

Cephalexin, crospovidone, color and colloidal silicon dioxide are mixed and granulated with an aqueous solution of povidone. The resulting granules are mixed with microcrystalline cellulose, crospovidone, colloidal silicon dioxide, and mannitol for 20 minutes. To this blend aspartame, D&C yellow 10 Aluminum Lake, magnesium stearate, flavour peppermint 517 and flavour fruit gum 912 are added. The resulting blend is then compressed.

The tablets made per the above example were subjected to accelerated stability studies at 40° C./75%RH, with the data showing no change in assay, friability (<1%) or disintegration time.

Therefore, the dispersible tablets of the present invention are not only stable at accelerated stability testing conditions but also are robust and can withstand mechanical stress during packaging and transport.

A comparative, randomized two-way crossover bioavailability study was conducted on the cephalexin 250 mg dispersible tablet (prepared as per the above example) formulation (T) and the commercially available cephalexin (250 mg/5 mL) suspension formulation (R) of Eli Lilly in 34 healthy volunteers under fasting conditions. The pharmacokinetic data obtained was analyzed at the 90% confidence interval (T/R) and the ratio of the least square means T/R (%) was calculated and is given in Table 1.

TABLE 1
Cephalexin dispersible tablet bio-profile in
comparison to cephalexin oral suspension

| | AUC₀₋ₜ | AUC_{0-∞} | Cₘₐₓ |
|---|---|---|---|
| Ratio¹ | 99.85% | 99.85% | 100.30% |
| 90% | 97.81 to 101.93 | 97.86% to 01.88% | 95.06% to 105.84% |
| Geometric | | | |
| C.I.² | | | |
| Intra- | 5.03% | 4.90 | 13.13% |
| Subject | | | |
| C.V. | | | |

¹calculated using least-square means according to the formula: e^{(T-R)} × 100
²90% Geometric confidence Interval using [symbol] n transformed data.

The results of the study showed that the 250 mg dispersible tablets of the present invention are bio-equivalent to cephalexin 250 mg/5 mL oral suspension under fasting conditions.

1. A process for the preparation of water-dispersible tablets of cephalexin, wherein the tablets disintegrate within 3 minutes in water at 20° C.±5° C. to form a uniform suspension, comprising granulating cephalexin, disintegrant and colloidal silicon dioxide with binder solution to form granules; drying the resulting granules; mixing the dried granules with disintegrant(s), fillers, lubricating agents and optionally other excipients; and compressing to form tablets.

2. The process according to claim 1 wherein the granules comprise a suspending agent and/or coloring agent.

3. The process according to claim 1 wherein other optional excipients comprise one or more of antiadherants, sweeteners, coloring agents and flavoring agents.

4. The process according to claim 1 where cephalexin is present as monohydrate.

5. The process according to claim 1 wherein cephalexin has a particle size d₉₀ less than 250 μm.

6. The process according to claim 1 wherein granulation is the wet granulation method.

7. The process according to claim 1 wherein the disintegrant(s) are selected from sodium starch glycolate, carboxy methylcellulose, croscarmellose sodium and crospovidone and combinations thereof.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. The process according to claim 2 wherein the suspending agent is selected from microcrystalline cellulose, sodium carboxy methylcellulose, colloidal silicon dioxide, mannitol, povidone, sodium starch glycolate or a combination thereof.

20. (canceled)

21. The process according to claim 2 wherein the suspending agent is present in an amount of about 0.25% to about 6.0% by weight of the total tablet weight.

22. The process according to claim 3 wherein the coloring agent is D&C Yellow Aluminum Lake.

23. The process according to claim 3 wherein the antiadherant is colloidal silicon dioxide.

24. The process according to claim 3 wherein the sweetening agent is selected from sugars, saccharin or its salts, aspartame or combinations thereof.

25. (canceled)

26. The process according to claim 3 wherein sweetening agent is present in an amount of about 0.01% to about 2.0% by weight of total weight of tablet.

27. (canceled)

28. A water dispersible dosage form of cephalexin comprising an intragranular portion and an extragranular portion:
the intragranular portion comprising a pharmaceutically acceptable amount of cephalexin or its salts, a disintegrant(s), and a suspending agent(s); and
the extragranular portion comprising one or more pharmaceutically acceptable excipients.

29. The water dispersible dosage form of claim 28 wherein cephalexin is present as a monohydrate.

30. The water dispersible dosage form of claim 28 wherein cephalexin has a particle size of d₉₀ less than 250 μm.

31. The water dispersible dosage form of claim 28 wherein the pharmaceutically acceptable excipients comprise one or more of fillers, binders, lubricants, antiadherants, sweeteners, coloring agents, and flavoring agents.

32. (canceled)

33. (canceled)

34. (canceled)

35. (canceled)

36. (canceled)

37. The water dispersible dosage form of claim 31 wherein the binder is present in an amount from about 0.25% to about 4% by weight of total tablet weight.

38. The water dispersible dosage form of claim 31 wherein the filler comprises one or more of lactose, microcrystalline cellulose, mannitol, and combinations thereof.

39. (canceled)

40. (canceled)

41. (canceled)

42. (canceled)

43. (canceled)

44. (canceled)

45. (canceled)

46. The water dispersible dosage form of claim 28 wherein the suspending agent is present in an amount of about 0.25% to about 6.0% by weight of the total tablet weight.

47. (canceled)

48. (canceled)

49. (canceled)

50. (canceled)

51. (canceled)

52. (canceled)

53. A method of treating an infection in a human caused by microorganisms susceptible to cephalexin comprising providing cephalexin in the form of the water dispersible tablet of claim 29.
